(19) United States
(12) Patent Application Publication
(10) Pub. No.: US 2017/0238982 A1
(43) Pub. Date: Aug. 24, 2017

(54) Medical Screw

(71) Applicant: Kenneth L. Alicastro, Plainview, NY (US)

(72) Inventor: Kenneth L. Alicastro, Plainview, NY (US)

(21) Appl. No.: 15/413,511

(22) Filed: Jan. 24, 2017

Related U.S. Application Data
(60) Provisional application No. 62/298,116, filed on Feb. 22, 2016.

(51) Int. Cl. A61B 17/86; A61B 17/88

(52) U.S. Cl. CPC A61B 17/8615; A61B 17/863; A61B 17/8888

(57) ABSTRACT

A dual drive screw that includes a head and a shaft. The head has a top surface, a first recess in the top surface comprising a first drive and a second recess below the first recess comprising a second drive. The shaft extends from the head opposite the top surface and includes an exterior surface with a thread formed on at least part of the exterior surface. The first drive may be a star drive, a hex drive, a square drive, a 12-point flange, a double square, a double hex, a spline drive or any other drive that has an opening large enough for a device to pass through and engage the second drive in the second recess. A first device engages the first drive and rotates the screw in a clockwise direction. A second device engages the second drive and rotates the screw in a counter-clockwise direction for removal. The dual drive screw is preferably implanted in a person.

This application claims priority from provisional application Ser. No. 62/298,116, filed on Feb. 22, 2016, which is incorporated herein in its entirety.

### FIELD OF THE INVENTION

The present invention is a screw that is used in medical procedures. In particular, the present invention relates to a medical screw with two drives in the head of the screw (also referred to herein as a dual drive screw or a dual drive screw head).

### BACKGROUND OF INVENTION

Screws are used in various medical procedures to attach bones to implant devices or to other bones. Typically, the screws are made of titanium or stainless steel and the lengths vary according to the intended use. In many cases, the screws are temporary and are removed after the patient sufficiently heals. Serious problems can occur in the operating room when a procedure calls for the removal of hardware and a screw drive becomes damaged or stripped. This is a serious concern for any orthopedic surgeon who performs this type of procedure.

A screw that is stripped can result in multiple adverse effects. Surgery time is increased (sometimes up to an hour or more), additional anesthesia has to be administered to the patient and, most significantly, the bone and surrounding tissue incur trauma while the surgeon attempts to remove the stripped screw. There are several devices on the market today for the removal of damaged screws, but they have been used with limited success. Most of these devices try to engage the damaged area or they require the damaged area to be cleaned (smoothed out) before the device attempts to engage the surface using counter clockwise threads.

In the past, during medical procedures involving screws to hold, e.g., bones together, the drive on the head of the screw often times gets stripped upon insertion making it difficult to remove. Accordingly, there is a need for a medical screw that can be easily removed from a patient even after the primary screw drive is damaged and cannot be used.

### SUMMARY OF THE INVENTION

In accordance with the present invention, a dual drive screw, preferably used as a medical screw, is provided. The dual drive screw comprises, consists of or consists essentially of a head and a shaft. The head has a top surface, a first recess in the top surface comprising a first drive and a second recess below the first recess comprising a second drive. The shaft extends from the head opposite the top surface and includes an exterior surface with a thread formed on at least part of the exterior surface. The first drive may be a star drive, a hex drive, a square drive, a 12-point flange, a double square, a double hex, a spline drive or any other drive that has an opening large enough for a device to pass through and engage the second drive in the second recess. A first device engages the first drive and rotates the screw. Preferably, the first device rotates the screw in a clockwise direction. The second drive may be a reverse thread drive, a torx drive, a slot drive, a Phillips drive, a tri-wing, a spanner head drive or any drive that can be engaged by a device that can pass through the first recess for the first drive. A second device engages the second drive and rotates the screw. Preferably, the second device rotates the screw in a counter-clockwise direction for removal. The dual drive screw is preferably implanted in a person.

### BRIEF DESCRIPTION OF THE FIGURES

The preferred embodiments of the dual drive screw of the present invention, as well as other objects, features and advantages of this invention, will be apparent from the accompanying drawings wherein:

FIG. 1 is a side view with a cutaway view of the head for a screw with a dual drive head having a star drive for the first drive and a reverse threaded head in a sub-recess for the second drive.

FIG. 2 is an end view of the dual drive head shown in FIG. 1.

FIG. 3 is a side view with a cutaway view of the head for a screw with a dual drive head having a hex drive for the first drive and a reverse threaded head in a sub-recess for the second drive.

FIG. 4 is an end view of the dual drive head shown in FIG. 3.

FIG. 5 is a side view with a cutaway view of the head for a screw with a dual drive head having a notched hex post drive for the first drive and a reverse threaded head in the wall of the recess for the second drive.

FIG. 6 is an end view of the dual drive head shown in FIG. 5.

FIG. 7 is a side view of a device for engaging and rotating a notched hex post drive in a dual drive head.

FIG. 8 is an end view of the device for engaging and rotating a notched hex post drive shown in FIG. 7.

FIG. 9 is a side view of a device for engaging and rotating a left hand/reverse thread drive in a dual drive head as shown in FIG. 5.

FIG. 10 is a side view of the device for engaging and rotating a left hand/reverse thread drive shown in FIG. 9.

FIG. 11 is an end view of the device for engaging and rotating a left hand/reverse thread drive shown in FIG. 9.

FIG. 12 is a side view of a device for engaging and rotating a left hand/reverse thread drive in a dual drive head as shown in FIGS. 1 and 3.

FIG. 13 is a top view of a dual screw head having a square head drive and a recessed slot head drive.

FIG. 14 is a top view of a dual screw head having a square head drive and a recessed Phillips head drive.

FIG. 15 is a top view of a dual screw head having a square head drive and a recessed Torx head drive.

FIG. 16 is a top view of a dual screw head having a hex head drive and a recessed flat Torx drive.

FIG. 17 is a top view of a dual screw head having a hex head drive and a recessed flat slot drive.

FIG. 18 is a top view of a dual screw head having a hex head drive and a recessed flat tri-wing drive.

### DETAILED DESCRIPTION OF THE INVENTION

The present invention is a dual drive medical screw having a first drive in the head of the screw, such as any standard drive (hex, star, etc.). Countersunk below the first drive is a second drive, such as a left hand reverse thread, in a recessed portion of the head. If the first drive becomes stripped and it is desired to remove the screw, the second drive provides a means for extraction. Having the dual head drive provides redundancy for the removal of the screw.

The dual head screws have a recess below the first screw drive that is also referred to as a sub-recess (a separate section or chamber) of the screw head that has a second drive (such as counter clockwise threads) integrally formed in the screw head. The second drive (such as the threads of a counter clockwise drive) can be easily engaged by the surgeon to remove the stripped screw by rotating the screw in counter-clockwise direction after the device (e.g., a screwdriver) engages the second drive.

Stardrive and hexdrive recesses are currently the predominant choice of design for medical screws. However, the dual head screws are not limited to star drives and hexdrives and any number of different screw head drives can be used, for example, the drives shown in Table I. The choice of which drive to use for the first drive and which drive to use for the second drive is determined by the configuration of the drives and one skilled in the art would easily be able to select the appropriate drives. As a rule of thumb, the first drive must have an opening large enough to allow the device or tool for engaging the second drive to pass through the opening in the first drive and engage the second drive. The dual drive design allows a “second chance” at screw removal and thereby saves time and expense.

The dual drive design is not limited to medical screws. One skilled in the art would recognize that the dual drive design could be utilized for any type of screw. Many industries will benefit from this cost effective design, e.g., automotive, aircraft, aerospace, home and commercial construction. However, when the dual drive screw is used as a medical screw, it is preferably made of titanium or stainless steel. When the dual drive screw is used in non-medical applications, it can be made from any suitable metal, plastic, ceramic or composite material without limitation. One skilled in the art will appreciate that the inventive feature is the design of the dual drive head and not the material of construction.

As used herein, the terms “screw drive” and “drive” refer to a system used to turn a screw. It is the structural portion of the head of the screw that allows torque to be applied to rotate the screw. The shape of this structural portion is not intended to be a limitation of the invention and it is contemplated that a variety of different shapes can be used.

As used herein, the term “reverse threaded drive” means that the threads of the screwdriver and the recessed screw head are designed to operate in a counter clockwise direction to thread the screwdriver tip into the screw head. When the dual drive screw is installed using the first drive, the screw is typically turned in a clockwise direction. The recessed second drive is typically used to extract the screw in the event that the first drive is damaged and cannot be used. Typically, the recessed second drive is not used to install the screw and is typically used only when the primary drive is damaged.

The screws have two drives that share the same opening in the head of the screw. The first drive is located in the top surface of the screw head, preferably in a recess. The first drive is selected so that the size of the opening for the first drive is large enough to allow a device for operating the second drive to pass through the recess for the first drive and engage the second drive. For example, the first drive may be a hex drive, a square drive, a 12-point flange, a double square, a double hex or a spline drive. The openings for these drives are large enough so that a device can pass through and operate a second drive, such as a reverse thread drive, a torx drive, a slot drive, a Phillips drive, a tri-wing or a spanner head drive.

The dual drive screw uses one or two mating devices or tools, such as a screwdriver, for rotating the screw clockwise and/or counter clockwise. The drives in the screw head are categorized based on commonality, with the less common drives being classified as “tamper-resistant.” Most heads come in a range of sizes, typically distinguished by a number, such as “Phillips #00” or “Torx T5.” These sizes do not necessarily describe a particular dimension of the drive shape, but are often arbitrary designations in the same sense as a “Size 8” dress.

The different types of drives that can be used include slot, Phillips, square, Robertson, hex, 12-point flange, hex socket (Allen), torx, tri-wing torq-set, spanner head, clutch, double-square, triple square, polydrive, spline drive, double hex, Bristol, Pentalobe. A listing of different drives and their configurations are shown below in Table I.

TABLE I

| Head configuration | Type |
|---|---|
| [symbol] | Slot |
| [symbol] | Phillips (PH) |
| □ | Square |
| [symbol] | Robertson |
| [symbol] | Hex |
| [symbol] | 12-point flange |
| [symbol] | Hex socket |
| | (Allen) |
| [symbol] | Security hex |
| | socket (pin-in- |
| | hex-socket) |
| [symbol] | Torx (T & TX) |
| [symbol] | Security Torx |
| | (TR) |
| [symbol] | Tri-Wing |
| [symbol] | Torq-set |
| [symbol] | Spanner head |
| | (Snake-eye) (TH) |
| [symbol] | Clutch (A & G) |
| [symbol] | One-way |
| [symbol] | Double-square |
| [symbol] | Triple square |
| | (XZN) |
| [symbol] | Polydrive |
| [symbol] | Spline drive |
| [symbol] | Double hex |
| [symbol] | Bristol |
| [symbol] | Pentalobe |

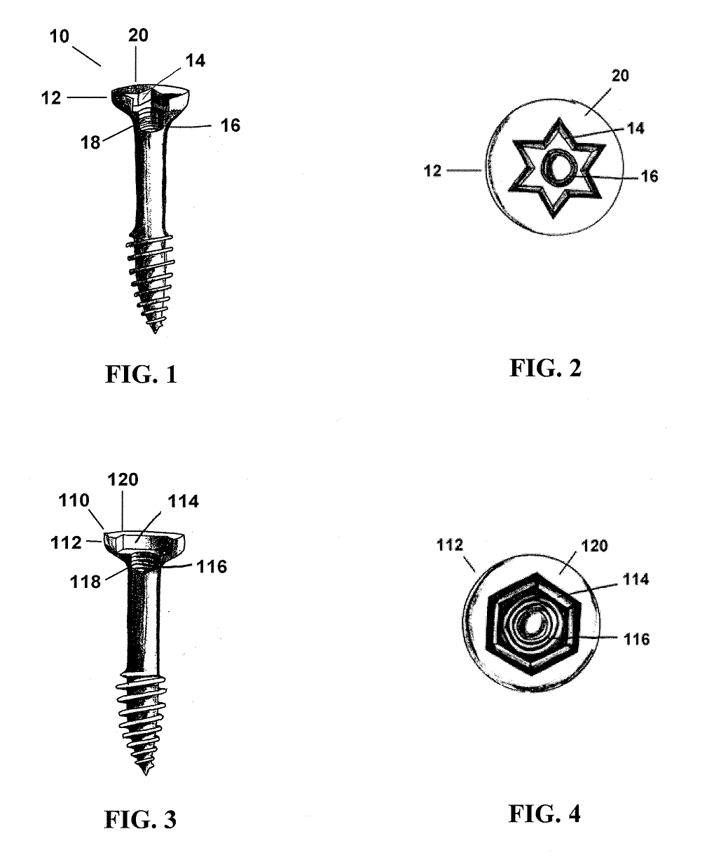

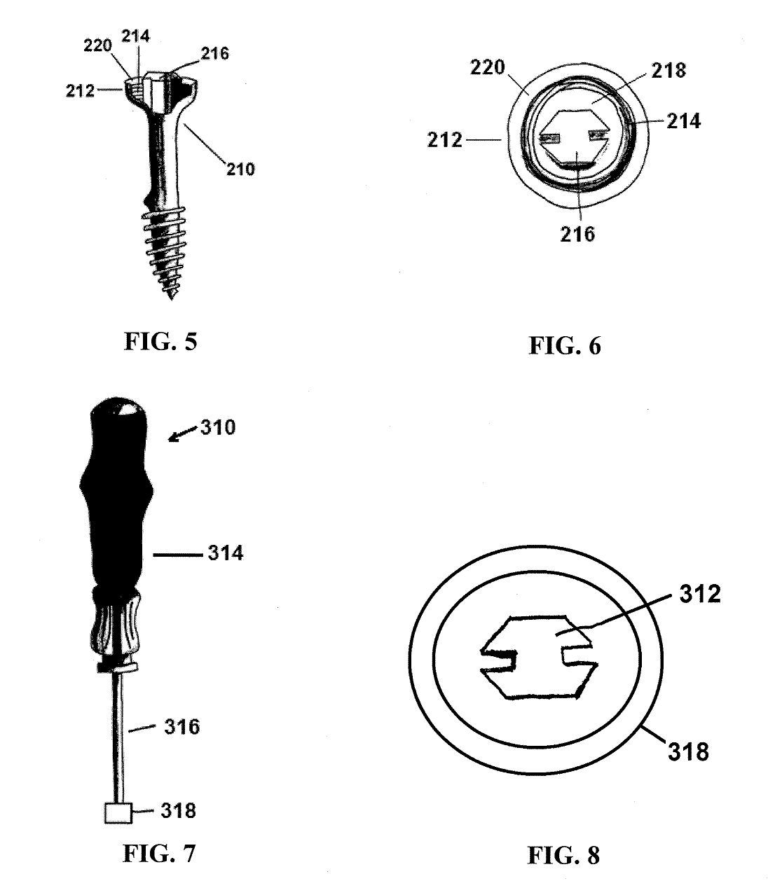

The medical screw will now be described with respect to the accompanying figures. FIGS. 1-6 show three embodiments of the medical screw. FIGS. 1 and 2 show the side view and top view, respectively, of a first embodiment of the dual drive medical screw 10 with the head 12 having a star drive 14 and a left hand/reverse thread drive 16 in the sub-recess 18 below the surface 20 of the head 12. FIGS. 3 and 4 show the side view and top view, respectively, of a second embodiment of the dual drive medical screw 110 with the head 112 having a hex drive 114 and a left hand/reverse thread drive 116 in the sub-recess 118 below the surface 120 of the head 112. FIGS. 5 and 6 show the side view and top view, respectively, of a third embodiment of the dual drive medical screw 210 with the head 212 having a notched hex post drive 216 and a left hand/reverse thread drive 214 in the wall of the recess below the surface 220 of the head 212.

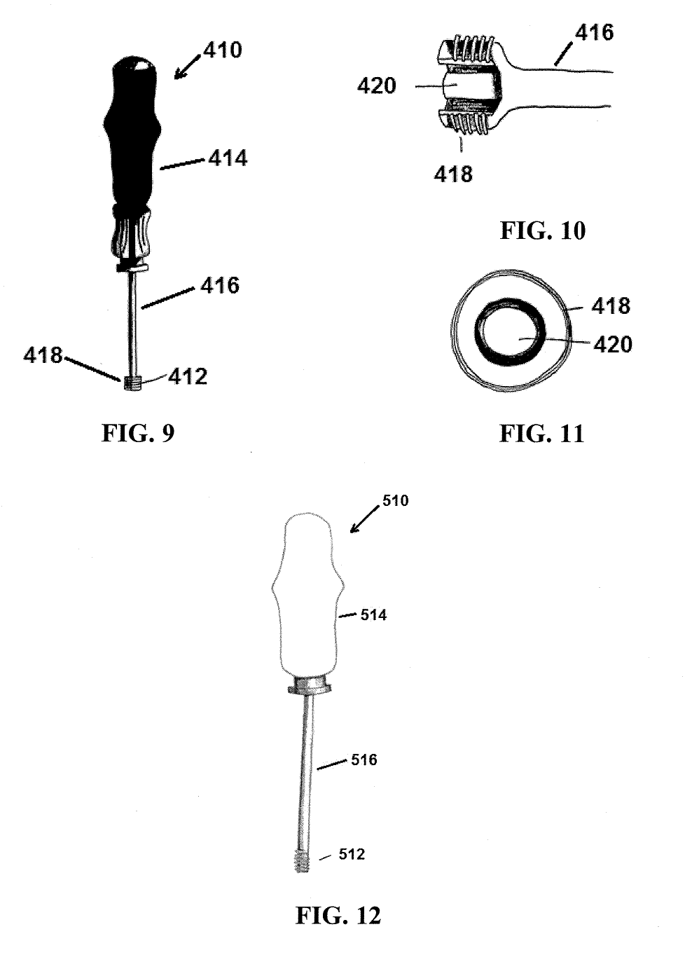

FIGS. 7 and 8 show the side view and an end view, respectively, of a device 310 for engaging and rotating a dual drive screw head with a notched hex drive 312. The device 310 has a handle 314, a driver shaft 316 and a recessed notched hex drive 312 with a smooth outer wall 318. FIGS. 9-11 show a device 410 for engaging and rotating a dual drive screw head with an external left hand/reverse thread drive 412. FIG. 9 shows a side view and FIGS. 10 and 11 show a side view and an end view, respectively, of the device that engages the reverse thread drive 412. The device 410 has a handle 414, a drive shaft 416 and a reverse thread drive 412 with a plurality of threads 418. FIG. 10 shows a cut-away side view of the external left hand/reverse thread drive 412 with a recess 420 at the distal end for receiving a notched head post (not shown). FIG. 12 shows an extraction screw driver 510 which has a reverse threaded end 512 opposite the handle 514. The handle 514 is connected to a drive shaft 516 with a distal end formed by the reverse threaded end 512. The reverse threaded end 512 is adapted to be threaded into the second drive of a screw head to remove the screw.

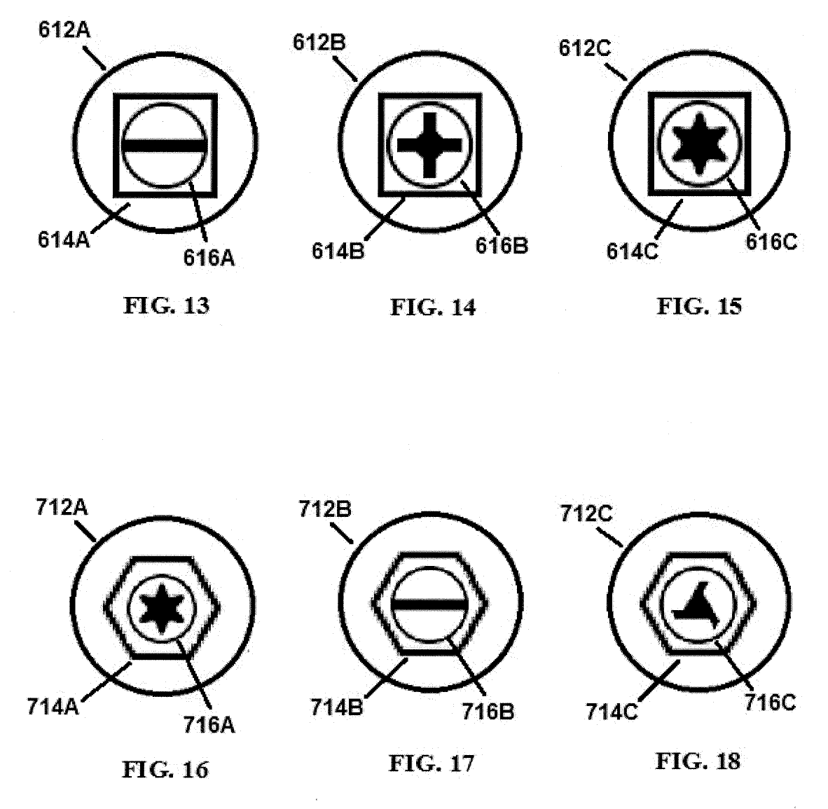

FIGS. 13-18 show top views of screw heads 612A-C and 712A-C with different dual drive designs. FIGS. 13-15 show screw head 612A-C designs where the first drive is a square drive 614A-C and the second (sub-recess) drives are slot, Phillips and torx drives 616A-C, respectively. FIGS. 16-18 show screw head 712A-C designs where the first drive is a hex drive 714A-C and the second (sub-recess) drives are torx, slot, tri-wing drives 716A-C, respectively.

Thus, while there have been described the preferred embodiments of the present invention, those skilled in the art will realize that other embodiments can be made without departing from the spirit of the invention, and it is intended to include all such further modifications and changes as come within the true scope of the claims set forth herein.

We claim:

1. A dual drive screw comprising:
a head with a top surface, a first recess in the top surface comprising a first drive and a second recess below the first recess comprising a second drive; and
a shaft extending from the head opposite the top surface, the shaft having an exterior surface with a thread formed on at least part of the exterior surface.

2. The dual drive screw according to claim 1, wherein, the first drive is a star drive, a hex drive, a square drive, a 12-point flange, a double square, a double hex or a spline drive.

3. The dual drive screw according to claim 2, wherein a first device is used to engage the first drive and rotate the screw.

4. The dual drive screw according to claim 3, wherein the first device rotates the screw in a clockwise direction.

5. The dual drive screw according to claim 1, wherein the second drive is a reverse thread drive, a torx drive, a slot drive, a Phillips drive, a tri-wing or a spanner head drive.

6. The dual drive screw according to claim 5, wherein a second device is used to engage the second drive and rotate the screw.

7. The dual drive screw according to claim 6, wherein the second device rotates the screw in a counter-clockwise direction.

8. The dual drive screw according to claim 1, wherein the dual drive screw is implanted in a person.

9. A dual drive screw comprising:
a head with a top surface, a first recess in the top surface comprising a first drive and a second recess below the first recess comprising a second drive, wherein, the first drive is a star drive, a hex drive, a square drive, a 12-point flange, a double square, a double hex or a spline drive and wherein the second drive is a reverse thread drive, a torx drive, a slot drive, a Phillips drive, a tri-wing or a spanner head drive; and
a shaft extending from the head opposite the top surface, the shaft having an exterior surface with a thread formed on at least part of the exterior surface.

10. The dual drive screw according to claim 9, wherein a first device is used to engage the first drive and rotate the screw in a clockwise direction.

11. The dual drive screw according to claim 10, wherein a second device is used to engage the second drive and rotate the screw in a counter-clockwise direction.

12. The dual drive screw according to claim 11, wherein the dual drive screw is implanted in a person.

13. A dual drive screw comprising:
a head with a top surface, a first recess in the top surface comprising a first drive and a second recess below the first recess comprising a second drive, wherein, the first drive is a star drive, a hex drive, a square drive, a 12-point flange, a double square, a double hex or a spline drive and wherein the second drive is a reverse thread drive, a torx drive, a slot drive, a Phillips drive, a tri-wing or a spanner head drive; and
a shaft extending from the head opposite the top surface, the shaft having an exterior surface with a thread formed on at least part of the exterior surface,
wherein a first device is used to engage the first drive and rotate the screw in a clockwise direction and a second device is used to engage the second drive and rotate the screw in a counter-clockwise direction, and wherein the dual drive screw is implanted in a person.
